(19) United States
(12) Patent Application Publication
(10) Pub. No.: US 2010/0233697 A1
(43) Pub. Date: Sep. 16, 2010

(54) METHOD FOR STANDARIZING SURFACE BINDING OF A NUCLEIC ACID SAMPLE FOR SEQUENCING ANALYSIS

(75) Inventors: Richard Joseph, Stoughton, MA (US); James J. DiMeo, Needham, MA (US)

Correspondence Address: LANDO & ANASTASI, LLP, ONE MAIN STREET, SUITE 1100, CAMBRIDGE, MA, 02142, US

(73) Assignee: HELICOS BIOSCIENCES CORPORATION, Cambridge, MA (US)

(21) Appl. No.: 12/618,251

(22) Filed: Nov. 13, 2009

Related U.S. Application Data
(60) Provisional application No. 61/114,872, filed on Nov. 14, 2008.

(51) Int. Cl. C12Q 1/68

(52) U.S. Cl. 435/6

(57) ABSTRACT

Methods are described which enable nucleic acid sample standardization prior to anchoring to a surface, especially useful in single molecule nucleic acid sequencing applications when sample is limiting or unamplified.

### CROSS-REFERENCE TO RELATED APPLICATIONS

This application claims priority to U.S. Patent Application Ser. No. 61/114,872, filed on Nov. 14, 2008, under 35 U.S.C. §119, the contents of which are hereby incorporated by reference in their entirety.

### SUMMARY

The present invention provides, at least in part, methods for sample performance analysis, e.g., for standardizing surface binding of a nucleic acid sample. The methods are useful for sequencing analysis (e.g., single molecule nucleic acid sequencing applications when limiting quantities of a sample are present, or a nucleic acid is not amplified).

Accordingly, in one aspect, the invention features a method for sample performance analysis, e.g., for standardizing surface binding of a nucleic acid sample. The method includes: (a) attaching an enzyme (e.g., a horseradish peroxidase or alkaline phosphatase), directly or indirectly to a nucleic acid; (b) anchoring the nucleic acid to a surface; (c) adding a substrate; (d) determining the amount of substrate enzymatically converted to a detectable product; and (e) comparing the product produced compared to a nucleic acid standard, thereby calibrating the nucleic acid performance relative to a standard.

In one embodiment, the enzyme is directly covalently attached to the nucleic acid.

In another embodiment, the enzyme is indirectly attached to the nucleic acid, e.g., via a binding pair. For example, the binding pair can be a biotin:streptavidin pair, a hapten/antibody pair, or a receptor:ligand pair. The binding pair can have a first member and a second member. In one embodiment, the first member of the binding pair is attached to the nucleic acid through a terminating nucleotide, e.g., a terminating nucleotide labeled with biotin or lacking a 3′-OH. In another embodiment, the second member of the binding pair is labeled with an enzyme, e.g., streptavidin.

In one embodiment, the nucleic acid is anchored, e.g., via hybridization or polymerase, directly or indirectly to the surface. For example, the surface can have an anchored oligonucleotide, e.g., oligo(T), capable of hybridizing at least in part to the nucleic acid.

In one embodiment, the surface is beads, magnetic beads, wells of a microplate or reaction sites on a planar surface.

In one embodiment, the substrate can produce a chromogenic or fluorescent detectable product in the presence of the enzyme, e.g., a horseradish peroxidase or alkaline phosphatase. For example, the substrate can be a chromogenic substance, such as 3,3′,5,5′-Tetramethyl benzidine (TMB) or umbelliferon phosphate, In another embodiment, the substrate can produce a detectable precipitate on the surface.

In one embodiment, the standard is a nucleic acid which attaches to a sequencing surface at a known density. For example, the surface can have individually optically resolvable single molecules, or colonies wherein the colonies are individually optically resolvable. The surface can be used for single molecule sequencing, e.g., sequencing by synthesis, ligation, or hybridization.

All publications, patent applications, patents, and other references mentioned herein are incorporated by reference in their entirety.

Other features, objects, and advantages of the invention will be apparent from the description and drawings, and from the claims.

### DETAILED DESCRIPTION

Many biological assays have been developed which apply a biological sample or material to a surface for further analysis. The biological material may be proteins, nucleic acids, carbohydrates, lipids, drugs, ligands, etc. The biological material may or may not have to have been modified in some way before anchoring to the surface. For example, for nucleic acid sequencing, the following steps might provide an example of a process:

a. the nucleic acid is isolated from a cellular source;

b. nucleic is fragmented;

c. the fragments are modified to attach a common sequence;

d. the common sequence is hybridized to a reverse complement sequence oligonucleotide on the surface;

e. the surface oligonucleotide functions as a primer in a template dependent, polymerase extension of the primer using labeled nucleotides.

One of the problems with these assays is that despite best efforts, the yield varies from sample-to-sample and day-to-day. Processing of a sample that varies widely through a series of steps results in overall low throughput, failed experiments, and high costs. Moreover, many samples can only be obtained in very limited quantities, for example, samples of human fluids, tissues or cells. Most of the sample is required for the direct analysis of the analyte by the method desired and in many cases aliquots can not be routinely sacrificed for real-time quality control analysis. What is needed is a rapid, high sensitivity assay which consumes little sample and provides results in a timely manner before running a detailed analysis on the entire sample.

Methods for nucleic acid sequencing have been developed recently which perform sequencing by synthesis on a single molecule level. Integral to such process is the anchoring of nucleic acids to a surface. The anchoring may be, for example, direct anchoring by means of a covalent bond or indirect anchoring via a binding pair, e.g., a biotin:streptavidin pair. The process of isolating, modifying, and hybridizing is highly variable. What is needed is a method which is able to quickly evaluate the integrity of each sample and allow the researcher enough information to adjust as necessary reaction parameters to normalize surface loading parameters from sample to sample. For example, a specific assay to be able to either pass/fail samples for further analysis or even adjust concentrations of samples to permit uniform, robust anchoring to a surface. The surface is one in which there is many discrete reaction sites or channels wherein each gets a different sample.

An example of a single molecule sequencing process follows. Epoxide-coated glass slides are prepared for oligo attachment. Epoxide functionalized 40 mm diameter #1.5 glass cover slips (slides) are obtained from Erie Scientific (Salem, N.H.). The slides are preconditioned by soaking in 3×SSC for 15 minutes at 37° C. Next, a 500-pM aliquot of 5′ aminated capture oligonucleotide (oligo dT(50)) is incubated with each slide for 30 minutes at room temperature in a volume of 80 ml. The slides are then treated with phosphate (1 M) for 4 hours at room temperature in order to passivate the surface. Slides are then stored in 20 mM Tris, 100 mM NaCl, 0.001% Triton® X-100, pH 8.0 at 4° C. until they are used for sequencing.

For the illustration of the sequencing process, see, e.g., U.S. patent application Ser. Nos. 12/043,033 (Xie et al. filed Mar. 5, 2008) and 12/113,501 (Xie et al. filed May 1, 2008) (e.g., FIGS. 1A and 1B). For sequencing, the slide is assembled into a 25 channel flow cell using a 50-μm thick gasket. The flow cell is placed into a Heliscope™ Sample Loader (Helicos BioSciences Corporation). A passive vacuum is built into the apparatus and is used to pull fluid across the flow cell. The flow cell is then rinsed with 150 mM HEPES/150 mM NaCl, pH 7.0 (“HEPES/NaCl”) and equilibrated to a temperature of 50° C. Separately, the nucleic acid to be sequenced is sheared to approximately 200-500 bases (Covaris), polyA tailed (50-70 ave. number dA's) using dATP and terminal transferase (NEB), 3′-end labeled with Cy5-ddUTP (PerkinElmer), and then diluted in 3×SSC to a final concentration of approximately 200 pM. A 100-μL aliquot is placed in one or more channels of the flow cell and incubated on the slide for 15 minutes. After incubation, the temperature of the flow cell is then reduced to 37° C. and the flow cell is rinsed with 1×SSC/150 mM HEPES/0.1% SDS, pH 7.0

(“SSC/HEPES/SDS”) followed by HEPES/NaCl. The resulting slide contains the primer template duplex randomly bound to the glass surface. Since the polyA/oligoT sequences are able to slide, the primer templates are filled and locked by firstly incubating the surface with Klenow exo+, TTP, in reaction buffer (NEB), washing thoroughly with HEPES/NaCl, and then incubating with Klenow exo+, dATP/dCTP/dGTP, in reaction buffer (NEB). A single step fill and lock can be done by incubating a mixture of TTP and 3 reversible terminator analogs of C, G, and A, see Virtual Terminator™ citations below. The slide is washed thoroughly again using the HEPES/NaCl to remove all traces of the dNTPs before initiating the actual sequencing by synthesis process. The temperature of the flow cell is maintained at 37° C. for sequencing and the objective is brought into contact with the flow cell.

Further, Virtual Terminator™ nucleotide analogs of 2′-deoxycytosine triphosphate, 2′-deoxyguanidine triphosphate, 2′-deoxyadenine triphosphate, and 2′ deoxyuracil triphosphate, each having a cleavable cyanine-5 label (at the 7-deaza position for ATP and GTP and at the C5 position for CTP and UTP, see, e.g., U.S. patent application Ser. Nos. 11/1803,339 (Siddiqi et al. filed May 14, 2007) and 11/603,945 (Siddiqi et al. filed Nov. 22, 2006), are stored separately in the buffer containing 20 mM Tris-HCl, pH 8.8, 50 μM MnSO₄, 10 mM (NH₄)₂SO₄, 10 mM KCl, 10 mM NaCl and 0.1% Triton X-100, and 50 U/mL Klenow exo-polymerase (NEB).

Sequencing proceeds as follows. The flow cell is placed on a movable stage that is part of a high-efficiency fluorescence imaging system Heliscope™ Single Molecule Sequencer (Helicos BioSciences Corporation). First, initial imaging is used to determine the positions of duplex on the epoxide surface. The Cy5 label attached to the nucleic acid template fragments is imaged by excitation using a laser tuned to 635 nm radiation in order to establish duplex position. For each slide only single fluorescent molecules that are imaged in this step are counted. Next, the cyanine-5 label is cleaved off incorporated template by introduction into the flow cell of 50 mM TCEP/250 mM Tris, pH 7.6/100 mM NaCl/TCEP solution”) for 5 minutes, after which the flow cell is rinsed with SSC/HEPES/SDS and HEPES/NaCl. The template is capped with 50 mM iodoacetamide/100 mM Tris, pH 9.0/100 mM NaCl (“Iodoacetamide solution”) for 5 minutes followed by rinse with SSC/HEPES/SDS and HEPES/NaCl. Imaging of incorporated nucleotides as described below is accomplished by excitation of a cyanine-5 dye using a 635-nm radiation laser. 100 nM Cy5-dCTP is placed into the flow cell and exposed to the slide for 2 minutes. After incubation, the slide is rinsed in SSC/HEPES/SDS, followed by HEPES/NaCl. An oxygen scavenger containing 30% acetonitrile and scavenger buffer (100 mM HEPES, 67 mM NaCl, 25 mM MES, 12 mM Trolox, 5 mM DABCO, 80 mM glucose, 5 mM NaI, and 0.1 U/μL glucose oxidase (USB), pH 7.0) is next added. The slide is then imaged (100-1000 frames) for 50-100 milliseconds at 635 nm. The positions having detectable fluorescence are recorded. After imaging, the flow cell is rinsed with SSC/HEPES/SDS and HEPES/NaCl. Next, the cyanine-5 label is cleaved off incorporated dCTP by introduction into the flow cell of TCEP solution for 5 minutes, after which the flow cell is rinsed with SSC/HEPES/SDS and HEPES/NaCl. The remaining nucleotide is capped with iodoacetamide solution for 5 minutes followed by rinse with SSC/HEPES/SDS and HEPES/NaCl. Optionally, the scavenger is applied again in the manner described above, and the slide is again imaged to determine the effectiveness of the cleave/cap steps and to identify nonincorporated fluorescent objects.

The procedure described above is then conducted with 100 nM Cy5-dATP, followed by 100 nM Cy5-dGTP, and finally 100 nM Cy5-dUTP. Uridine may be used instead of Thymidine due to the fact that the Cy5 label is incorporated at the position normally occupied by the methyl group in Thymidine triphosphate, thus turning the dTTP into dUTP. The procedure (expose to nucleotide, polymerase, rinse, scavenger, image, rinse, cleave, rinse, cap, rinse, scavenger, final image) is repeated for a total of 80-120 cycles.

Once the desired number of cycles is completed, the image stack data (e.g., the single-molecule sequences obtained from the various surface-bound duplexes) are aligned to produce the individual sequence reads. The individual single molecule sequence read lengths obtained range from 2 to 50+ consecutive nucleotides. Only the individual single molecule sequence read lengths above some predetermined cut-off depending upon the nature of the sample, e.g., greater than 20 bases and above, are analyzed.

The most critical factor to a successful experiment is the attachment of the sample of interest to the surface. Too much anchoring of the sample to the surface results in low or no yield of reads since the single molecules are spaced too closely to be uniquely resolved during analysis. Too little sample anchored results in reduced throughput and potentially jeopardizes the analysis outcome due to little data obtained. The concept of certain embodiments described herein describes a methodology for analyzing sample integrity and performance before initiating the single molecule process. The analysis is rapid and accurately predicts the performance of a sample before committing time, resources and costly materials to the single molecule analysis process.

As described hereinabove (page 4, line 13), the sample is “3′-end labeled with Cy5-ddUTP” which demonstrates a potential method, e.g., determine amount of Cy5 fluorescence associated with the sample, which might be used for evaluating the sample, however the analysis must be performed on a single molecule platform in order to determine the efficacy with which the sample hybridizes to the surface. Additionally, the sequencing instrument as currently configured is desirably fully loaded with reagents in order to perform the analytical evaluation of the hybridization process. Information about the integrity or performance of the sample loaded into a channel in the flow cell might not be available for several days following start of the run.

The method described modifies the process in the example on page 4 to enable analytical evaluation of the performance of the sample using reagents and instruments found in many molecular biology labs and not dependent on single molecule detection platforms. For example, the method for sample performance analysis comprises:
a. attaching an enzyme directly or indirectly to a nucleic acid;
b. anchoring nucleic acid to a surface;
c. adding a substrate;
d. determining the amount of substrate enzymatically converted to detectable product; and
e. comparing the product generated compared to a nucleic acid standard, thereby calibrating the nucleic acid performance relative to a standard.

The example describes using an enzyme catalyzed reaction to determine sample integrity before applying sample(s) to a single molecule platform. One example is to utilize a hapten labeled ddNTP, e.g. biotin-ddATP or biotin-ddTTP, in which to block the 3′-ends of the nucleic acid sample. The nucleic acid can then be hybridized to a surface, the biotin detected and measured using a streptavidin coupled to an enzyme, such as horseradish peroxidase or alkaline phosphatase, and adding a substrate which generates either a chromogenic or fluorescent product which can be detected. The relative amount of product formed is compared to a standard nucleic acid sample which has a known level of performance.

The assay can be set up in many different formats, for example, the enzyme can be directly covalently attached to the nucleic acid or indirectly attached to the nucleic acid by means of a binding pair. The binding pair can be biotin:streptavidin, hapten/antibody, or receptor:ligand. The first member of the binding pair can be attached to the 3′ end of the nucleic acid through a terminating nucleotide, for example biotin-ddNTP, digoxigenin-ddNTP, or dye-ddNTP or even similar analogs of dNTP/NTPs. In a preferred example, the terminating nucleotide lacks a 3′-OH. Examples of terminating nucleotides include, but are not limited to: 2′,3′-dideoxynucleotides, 3′-deoxynucleotides, acyclonucleotides, 3′-amino or 3′-azido nucleotides. The second member of the binding pair is labeled with an enzyme. Examples of the second member of the binding pairs are streptavidin, anti-digoxigenin, anti-dye (e.g., anti-fluorescein or other anti-dye available from InVitrogen). The enzyme may be for example horseradish peroxidase or alkaline phosphatase.

The nucleic acid can be anchored directly or indirectly to the surface. The surface, for example, may take the form of beads, magnetic beads, wells of a microplate or reaction sites on a planar surface or a multi-dimensional surface. For example if the nucleic acid has a 5′-amine direct attachment to an epoxide surface can be used. Preferably, the nucleic acid is anchored via hybridization wherein the surface has an oligonucleotide anchored capable of hybridizing at least in part to the nucleic acid. When the nucleic acid has attached a polyA sequence, surfaces having an oligo(T) are used. Alternatively, the nucleic acid can be anchored to the surface via a polymerase.

The enzyme substrate is capable of producing a chromogenic or fluorescent detectable product in the presence of the enzyme. The amount of product produced is directly proportional to amount of enzyme on the surface. The substrate may also result in a product which forms a detectable precipitate on the surface. For example, the enzyme might be horseradish peroxidase where the substrate is chromogenic TMB (3,3′,5,5′-tetramethyl benzidine). Alternatively, the enzyme is alkaline phosphatases and the substrate is umbelliferon phosphate which generates fluorescent umbelliferon. In cases where sample is extremely limiting in amount or concentration, enzyme amplification methods may also be used, for example, the Tyramide Signal Amplification (TSA) available from PerkinElmer: (http://las.perkinelmer.com/applicationssummary/applications/TSA+-+Main.htm).

The signal detectable from the nucleic acid sample is compared to the signal obtained from a standard, control nucleic acid which attaches to a sequencing surface at a known density. The surface density may be measured in individually optically resolvable single molecules. Optionally, the surface density is measured as colonies wherein the colonies are individually optically resolvable.

The nucleic acid to be anchored to a surface can be analyzed by nucleic acid sequencing. The nucleic acid can be DNA, RNA, unamplified or amplified. The preferred analysis is the surface is used for single molecule sequencing wherein the sequencing is sequencing by synthesis. Optionally, the method of sequencing is sequencing by ligation or sequencing by hybridization.

When introducing elements of the examples disclosed herein, the articles “a,” “an,” “the” and “said” are intended to mean that there are one or more of the elements. The terms “comprising,” “including” and “having” are intended to be open-ended and mean that there may be additional elements other than the listed elements. It will be recognized by the person of ordinary skill in the art, given the benefit of this disclosure, that various components of the examples can be interchanged or substituted with various components in other examples.

All references cited herein are incorporated herein by reference in their entirety and for all purposes to the same extent as if each individual publication or patent or patent application was specifically and individually indicated to be incorporated by reference in its entirety for all purposes.

### EQUIVALENTS

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

We claim:

1. A method for sample performance analysis which comprises:
a. attaching an enzyme directly or indirectly to a nucleic acid;
b. anchoring nucleic acid to a surface;
c. adding a substrate;
d. determining the amount of substrate enzymatically converted to detectable product; and
e. comparing the product produced compared to a nucleic acid standard, thereby calibrating the nucleic acid performance relative to a standard.

2. The method of claim 1, wherein the enzyme is directly covalently attached to the nucleic acid.

3. The method of claim 1, wherein the enzyme is indirectly attached to the nucleic acid.

4. The method of claim 3, wherein the indirect attachment is via a binding pair.

5. The method of claim 4, wherein the binding pair is a biotin:streptavidin pair, a hapten/antibody pair, or a receptor:ligand pair.

6. The method of claim 4, wherein the first member of the binding pair is attached to the nucleic acid through a terminating nucleotide.

7. The method of claim 6, wherein the terminating nucleotide is labeled with biotin.

8. The method of claim 6 wherein the terminating nucleotide lacks a 3′-OH.

9. The method of claim 4, wherein the second member of the binding pair is labeled with an enzyme.

10. The method of claim 9, wherein the second member of the binding pair is streptavidin.

11. The method of claim 1, wherein the enzyme is horseradish peroxidase or alkaline phosphatases.

12. The method of claim 1, wherein the nucleic acid is anchored directly or indirectly to the surface.

13. The method of claim 12, wherein the nucleic acid is anchored via hybridization.

14. The method of claim 13, wherein surface has an oligonucleotide anchored capable of hybridizing at least in part to the nucleic acid.

15. The method of claim 14, wherein the surface anchored oligonucleotide is oligo(T).

16. The method of claim 12, wherein the nucleic acid is anchored to the surface via a polymerase.

17. The method of claim 1 wherein the surface is beads, magnetic beads, wells of a microplate or reaction sites on a planar surface.

18. The method of claim 1, wherein the substrate produces a chromogenic or fluorescent detectable product in the presence of the enzyme.

19. The method of claim 1, wherein the substrate produces a detectable precipitate on the surface.

20. The method of claim 18, wherein the enzyme is horseradish peroxidase and the substrate is chromogenic TMB (3,3′,5,5′-Tetramethyl benzidine).

21. The method of claim 18, wherein the enzyme is alkaline phosphatases and the substrate is umbelliferon phosphate.

22. The method of claim 1, wherein the standard is a nucleic acid which attaches to a sequencing surface at a known density.

23. The method of claim 22, wherein the surface has individually optically resolvable single molecules.

24. The method of claim 22, wherein the surface has colonies wherein the colonies are individually optically resolvable.

25. The method of claim 22, wherein the surface is used for single molecule sequencing.

26. The method of claim 25 wherein the sequencing is sequencing by synthesis, by ligation or by hybridization.
